(19) United States
(12) Patent Application Publication
(10) Pub. No.: US 2003/0031806 A1
(43) Pub. Date: Feb. 13, 2003

(54) Medicinal inhalation devices and components coated using thermal chemical vapor deposition

(76) Inventor: Philip A. Jinks, Loughborough (GB)

Correspondence Address: Office of Intellectual Property Counsel, 3M Innovative Properties Company, PO Box 33427, St. Paul, MN, 55133-3427, US

(21) Appl. No.: 10/192,333

(22) Filed: Jul. 10, 2002

Related U.S. Application Data
(60) Provisional application No. 60/304,109, filed on Jul. 10, 2001.

(51) Int. Cl. A61L 2/00; C23C 16/00

(52) U.S. Cl. 427/569; 427/2.1; 427/255.28

(57) ABSTRACT

A method of making a medicinal inhalation device with a fluorocarbon (CF₂)ₙ-type polymer thin film coating by pyrolyzing a monomer gas to produce polymerizable CF₂ species in the vicinity of the surface on which the fluorocarbon polymer film is to be formed, and maintaining the surface at a lower temperature than that of the heat source to induce deposition and polymerization of CF₂ species on the surface.

### FIELD OF THE INVENTION

The present invention relates to medicinal inhalation devices and components for such devices. The invention is particularly related to medicinal inhalation devices or components therefor having a surface coated with a fluorocarbon (CF₂)ₙ-type polymer thin film exhibiting enhanced chemical and mechanical stability and methods of making such devices and components.

### BACKGROUND OF THE INVENTION

Medicinal inhalation devices, including pressurized inhalers, such as metered dose pressurized inhalers (MDIs) and dry powder inhalers (DPIs), are widely used for delivering medicaments.

Medicinal inhalation devices typically comprise a plurality of hardware components, which can include for example gasket seals, valves including their individual components, such as valve stems, tanks, springs, retaining cups and seals; containers; actuators, as well as a number of internal surfaces which may be in contact with the medicinal formulation during storage or come in contact with the medicinal formulation during delivery. Often a desirable material for a particular component is found to be unsuitable in regard to its surface properties, e.g. surface energy, and/or its interaction with the medicinal formulation. For example, the relatively high surface energy of materials typically used in MDIs, e.g. acetal polymer for valve stems, or nitrile or neoprene elastomers for seals, or deep drawn stainless steels or aluminum for containers, can cause medicament particles in suspension formulations to adhere irreversibly to the surfaces of corresponding component(s), which has a consequent impact on the uniformity of medicinal delivery. Other examples of potentially undesirable interactions between a component and the medicinal formulation may include enhanced medicament degradation; adsorption of medicament or permeation of a formulation constituent, e.g. propellant, into seal materials; or extraction of chemicals from seal materials. Also the use of materials having relatively high surface energy for certain components, e.g. valves and the individual components of a valve, may have undesirable effects for the operation of movable components of a medicinal inhalation device.

In an attempt to reduce adhesion of suspended medicinal particles onto the wall of a container, EP 642992 discloses an aerosol container for pharmaceutically active aerosols, which are provided in the form of a suspension in the container; the suspension comprising at least a propellant gas, and the inside wall of the container is plastic coated. Polytetrafluorethylene (PTFE) and perfluoroethylenepropylene are disclosed as examples of materials for the plastic coating. It is disclosed that various procedures can be applied for the coating of the inner wall of the container, such as plasma coating, impregnating/spraying, hard anodization with PTFE deposition, chemical vapor deposition (CVD), physical vapor deposition as well as other procedures known in the art for this purpose. Plasma coating is disclosed to be especially preferred.

WO 96/32099, WO 96/32150, WO 96/32151, and WO 96/32345 disclose MDIs having part or all of its internal surfaces coated with one or more fluorocarbon polymers, optionally in combination with one or more non-fluorocarbon polymers for dispensing a drug suspension formulation of particular drugs. As fluorocarbon polymers are disclosed fluorocarbon polymers, which are made of multiples of one or more of the following monomeric units: tetrafluoroethylene (PTFE), fluorinated ethylene propylene (FEP), perfluoroalkoxyalkane (PFA), ethylene tetrafluoroethylene (ETFE), vinyldienefluoride (PVDF), and chlorinated ethylene tetrafluoroethylene. It is disclosed that fluorinated polymers, which have a relatively high ratio of fluorine to carbon, such as perfluorocarbon polymers e.g. PTFE, PFA, and FEP, are preferred. These documents disclose that the MDI can may be coated by the means known in the art of metal coating, either by pre-coating the coil stock before being stamped or drawn into the can shape or by coating preformed MDI cans by dipping the cans into the polymer or pouring polymer into cans or by electrostatic dry powder coating, spray coating, or plasma polymerization.

WO 99/42154 discloses an apparatus for dispensing a medicament, wherein at least a portion of one or more of the internal surfaces of components of the apparatus, which come into contact with medicament during storage or dispensing, has a layer of one or more cold plasma polymerized monomers bonded to at least a portion thereof. It is disclosed that the cold plasma treatment is a vacuum procedure in which the components are placed inside a chamber, which is evacuated, and after one or more monomers are introduced to the chamber, a 13.56 MHZ r.f. signal is applied to an external antenna. The plasma is ignited within the chamber and maintained for a given time at the pre-selected power setting, and at the end of the treatment the plasma is extinguished, and the products retrieved. As monomers for use in this process are disclosed perfluoro-cyclohexane, perfluoro-hexane, tetrafluoroethylene, trifluoroethylene, vinylidene fluoride, vinyl fluoride, or siloxanes.

WO 99/47195 discloses a valve for an aerosol container for dispensing a suspension or solution of a substance in a liquid propellant contained therein, wherein the valve comprises a valve body defining a chamber, a transfer passage through which a quantity of substance to be dispensed can pass from the container into the chamber, and dispensing means which allows the substance to be dispensed, in which the surface of the chamber is coated with a fluorinated material including fluorine coatings, plastics materials comprising fluorinated materials. It is disclosed that the fluorinated coating is preferably a plasma coating, for example, a CF4 plasma coating. In regard to the plasma coating it is disclosed that the components to be coated are placed inside a chamber which is evacuated; the fluorine monomer or fluorine source is introduced into the chamber at a controlled rate; the plasma is ignited within the chamber and maintained for a given time at a chosen power setting. At the end of the treatment the plasma is extinguished, the chamber flushed and the products retrieved.

WO 00/78286 discloses a medicinal aerosol steroid solution formulation product with enhanced chemical stability, comprising an aerosol container equipped with a dispensing valve and containing a medicinal aerosol formulation having a 20-ketosteroid drug dissolved therein, said 20-ketosteroid having an OH group at the C-17 or C-21 position or both, with the proviso that said 20-ketosteroid is other than flunisolide, and wherein said container is provided with a non-metal interior surface so as to reduce chemical degradation of the 20-ketosteroid. It is disclosed that the most preferred type of container for use in the present invention is a conventional aluminum (or aluminum alloy) aerosol canister, but with an interior coating of an inert material. As interior coating materials, epoxy-phenolic resins, epoxy-urea-formaldehyde resins, PTFE, and FEP are disclosed. It is disclosed that the coated containers can be made by pre-coating the metal roll stock before forming the container or by coating the container after it is made, by various techniques known in the art of coating, such as plasma coating, electrostatic dry powder coating, impregnating/spraying, hard anodization with polymer deposition, chemical vapor deposition (CVD), physical vapor deposition (PVD), as well as other procedures known in the art for this purpose.

### SUMMARY OF THE INVENTION

It has been found that the application of the fluorocarbon polymeric coatings onto component surfaces of medicinal inhalation devices via impregnating, spray coating, electrostatic dry powder coating or hard anodization suffers from a number of disadvantages. For example, it is often difficult to provide thin films (i.e. films having a thickness of 100 μm or less) and/or to coat surfaces of complex-formed components, such as components of valves, like valve stems, tanks (metering tanks) or springs.

Although film deposition via plasma coating, CVD or PVD has been proposed, with the exception of the plasma coating processes described in WO 99/42154 and WO 99/47195, no information concerning the actual process of depositing fluorocarbon polymer films onto component surfaces of medicinal inhalation devices is provided. It has been found that the application of PVD for the deposition of fluorocarbon polymeric films is difficult due to problems associated with e.g. the physical vaporization of an appropriate fluorocarbon source species for the subsequent film deposition. It also has been found that the resulting properties of fluorocarbon polymer films deposited through CVD or plasma polymerization depend on the particular process conditions and/or fluorine monomer source used. For example, plasma coating procedures as described in WO 99/42154 and WO 99/47195 (i.e. continuous plasma polymerization) typically produce fluorocarbon polymer films with a high degree of cross-linking (greater than 50%) resulting in film brittleness, which is undesirable for long term stability during storage and/or use in conjunction with movable components (i.e. components where a certain degree of movement or flexing occurs either during manufacture or use). Also such produced films, typically exhibiting sub-optimal film stoichiometry as well as significant concentrations of undesired moieties (greater than 70%) and defects, typically show chemical instability, as well as potentially reactive surface sites due to residual unpaired electrons from the polymerization process.

Until now there has been no recognition that the structural properties of a deposited fluorocarbon polymer film onto surfaces of components and/or internal surfaces of medicinal inhalation devices are critical, e.g. for the reduction of undesirable interactions between a component or its surface and the medicinal formulation or the enhancement the operation of movable components over the entire shelf-life of a medicinal inhalation device (typically 2-3 years). There is an ongoing need to provide a medicinal inhalation device having a surface coated with a fluorocarbon polymer thin film exhibiting enhanced chemical stability and inertness together with enhanced mechanical stability, as well as a method of making such a medicinal inhalation device. It is also desirable to provide medicinal inhalation device components comprising a surface coated with such a fluorocarbon polymer thin film as well as a method of making such components.

It has been found that the provision of medicinal inhalation device having a surface coated with a fluorocarbon (CF₂)ₙ-type (i.e. PTFE-type) polymer thin film having enhanced chemical and mechanical stability can be realized by forming the fluorocarbon polymer film through a thermal CVD method in which a monomer gas is pyrolyzed to generate a source of polymerizable CF₂ species.

Accordingly, the invention provides a method of making a medicinal inhalation device for delivery of a medicament or a medicinal formulation comprising a step of forming a fluorocarbon (CF₂)ₙ-type polymer thin film on a surface of the device, said forming step comprising the sub-steps of:

a) exposing a monomer gas to a source of heat having a temperature sufficient to pyrolyze the monomer gas, the monomer gas selected to produce upon pyrolysis a source of reactive species that includes polymerizable CF₂ species and that selectively promotes CF₂ polymerization, the reactive species source being in the vicinity of the surface on which the fluorocarbon polymer thin film is to be formed; and

b) maintaining the surface substantially at a temperature lower than that of the heat source to induce deposition and polymerization of the CF₂ species on the surface.

Preferably the medicinal inhalation device is a MDI or a DPI, more preferably a MDI. The (CF₂)ₙ-type polymer thin film is preferably formed on an internal surface of the device, more preferably on an internal surface that is to be in contact with a medicament or a medicinal formulation during storage or delivery from the medicinal inhalation device. Due to the desirable mechanical stability of the deposited (CF₂)ₙ-type films, it is also advantageous to form a (CF₂)ₙ-type polymer thin film on a surface that comes in contact with a movable component of the device or on a surface of a movable component of the device.

The method of forming the fluorocarbon polymer film can also be used for the preparation of individual components of a medicinal inhalation device. Accordingly, in another aspect, the invention provides a method of making a component for a medicinal inhalation device comprising a step of forming a fluorocarbon (CF₂)ₙ-type polymer thin film on a surface of the component, said forming step comprising the sub-steps a) and b) as described above. The component on which surface the film is formed may include any component used in a medicinal inhalation device, preferably a MDI or a DPI, more preferably a MDI. The component is preferably a movable component of such a device and/or a component having a surface which will come into contact with a movable component of such a device and/or a component having a surface which will come in contact with a medicament or a medicinal formulation during storage in or delivery from a medicinal inhalation device. Preferably, the monomer gas used in the methods in accordance with the invention includes hexafluoropropylene oxide. The heat source preferably is a resistively-heated conducting filament suspended over the surface or a heated plate having a pyrolysis surface that faces the surface on which the fluorocarbon polymer thin film is to be formed. The heat source temperature is preferably greater than about 500 K and the surface is preferably substantially maintained at a temperature less than about 300 K.

In other preferred embodiments of the methods, a first sub-step of applying plasma excitation power to the monomer gas is carried out. In an exemplary embodiment, the monomer gas is not substantially pyrolyzed during plasma excitation power application. In other preferred embodiments, the monomer gas is exposed to the heat source simultaneously with application of plasma excitation power to the monomer gas. Whenever plasma excitation power is applied, it preferably is characterized by an excitation duty cycle having alternating intervals in which excitation power is applied and in which no excitation power is applied to the monomer gas.

A method forming a (CF₂)ₙ-type (i.e. PTFE-type) polymer thin film on a surface of a structure comprising the steps of a) and b) is known from WO 97/42356. However said document is silent with respect to medicinal inhalation devices and components for such devices.

The methods in accordance with the invention advantageously provide medicinal inhalation devices or components therefor having a (CF₂)ₙ-type thin film on a surface of the medicinal inhalation device or on a surface of the component, respectively, wherein the film exhibits a high compositional CF₂ fraction in conjunction with a low degree of cross-linking density.

Thus, a further aspect of the invention is the provision of a medicinal inhalation device for delivery of a medicinal formulation comprising a fluorocarbon (CF₂)ₙ-type polymer thin film on a surface of the device, said film having a CF₂ fraction of at least 85% and a cross-linking density of less than about 20%. Preferably the medicinal inhalation device is a MDI or a DPI, more preferably a MDI.

An additional aspect of the invention is the provision of a component for a medicinal inhalation device comprising a fluorocarbon (CF₂)ₙ-type polymer thin film on a surface of said component; said film having a CF₂ fraction of at least 85% and a cross-linking density of less than about 20%.

In other preferred embodiments of the medicinal inhalation device and the component therefor, the film has a CF₂ fraction of at least 90% (more preferably at least 95%) and a cross-linking density of less than about 15% (more preferably less than about 10%). In other preferred embodiment of the medicinal inhalation device and the component therefor, the film has a thickness of about 0.0002 μm to about 100 μm, more preferably about 0.01 μm to about 3 μm.

Because the fluorocarbon (CF₂)ₙ-type polymer thin films of the medicinal inhalation devices and components in accordance with the invention have a high compositional CF₂ fraction and a low degree of cross-linking density, the films are flexible, i.e. non-brittle, and thus exhibit superior mechanical stability. This is especially desirable for the operation of movable or flexible components or assemblies, in particular valves and their individual components. The films also exhibit superior chemical stability and inertness, which in conjunction with enhanced mechanical stability is advantageous for long-term performance of the medicinal inhalation device as well as the reduction of undesirable interactions between a component or a surface of such a device and a medicinal formulation.

### BRIEF DESCRIPTION OF THE DRAWINGS

FIG. 1 is a schematic diagram of a vacuum chamber apparatus suitable for carrying out the film deposition processes.

FIG. 2 is a cross-sectional view of a preferred embodiment of a medicinal inhalation device provided in accordance with the invention.

FIG. 3 is a cross-sectional view of another preferred embodiment of a medicinal inhalation device provided in accordance with the invention.

FIG. 4 is a cross-sectional view of a third preferred embodiment of a medicinal inhalation device provided in accordance with the invention.

FIG. 5 is a cross-sectional view of a fourth preferred embodiment of a medicinal inhalation device provided in accordance with the invention.

### DETAILED DESCRIPTION

In the methods according to the invention, a surface of a medicinal inhalation device or a component for such a device to be coated with a (CF₂)-like thin film is exposed to a fluorocarbon monomer species, which is pyrolyzed through the application of a thermal input excitation to generate a source of polymerizable CF₂ species (thermal CVD process).

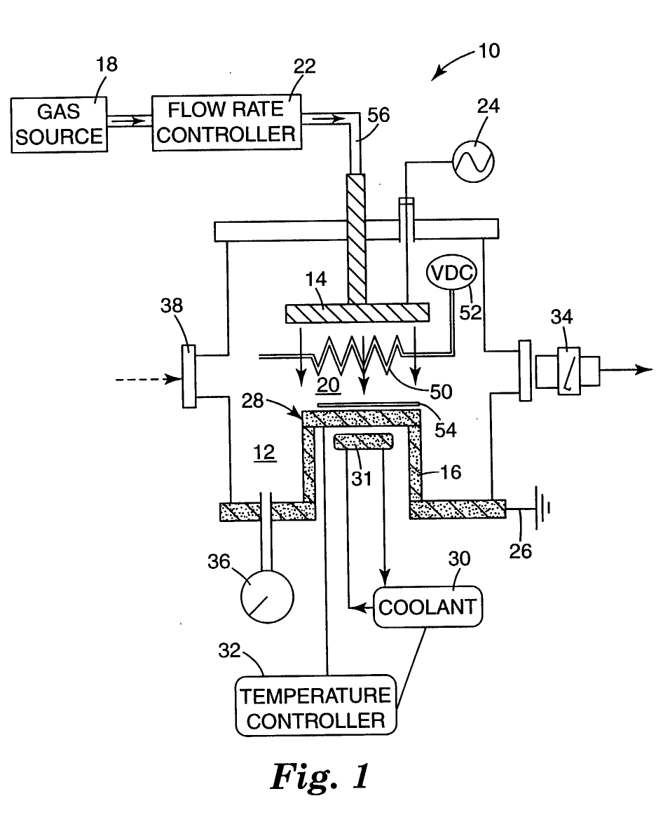

A vacuum deposition chamber like that schematically illustrated in FIG. 1 can be employed for carrying out the thermal CVD deposition process. The example deposition system 10 includes an air-tight vacuum chamber 12 formed of, e.g., steel, and includes a heated surface, e.g. a hot-filament 50. The hot-filament or other heated surface is preferably provided in a position relative to the input feed gas flow such that the input feed gas flows in the vicinity of the heated structure; whereby the gas is pyrolyzed to produce a source of reactive deposition species. For example, as shown in FIG. 1, a hot-filament 50 can be positioned just below an upper shower-head 14, which may be formed of e.g. aluminum. As shown in FIG. 1, the upper shower-head 14 may be an electrode connected electrically to a radio frequency (rf) power source 24, or other power source, but which need not be powered in this case. The hot-filament can be heated by, e.g., resistive heating. In this case, a dc voltage source 52 is provided to apply the heating voltage to the filament, consisting of, e.g., a Ni/Cr wire.

The upper shower-head 14 is preferably configured with connection to a feed gas source 18 such that the gas 20 injected to the chamber passes over the hot-filament, e.g. through tubes in a conventional the shower-head configuration. Preferably, the shower-head tubes provide a reasonably equal flow of gas per unit area of the upper electrode. Accordingly, the shower-head tubes should be spaced such that the concentration of the gas injected out of the shower-head is relatively uniform. The number and spacing of the tubes is dependent upon the specific pressure, temperature, and other process parameters, as will be recognized by those skilled in the art. For example, for a typical process employing a pressure of about 1 Torr, the shower-head tube spacing is about 1 cm.

A flow rate controller 22 is preferably provided to enable control of the flow of gas through the upper shower-head 14 into the chamber. A pump 34 is provided for evacuating the deposition chamber to a desired pressure; the pressure of the chamber is monitored by way of, e.g., a pressure gauge 36. Also preferably provided is an analysis port 36 for enabling a user to monitor progress of the deposition process.

The lower stage 16, which as shown in FIG. 1 may be a grounded electrode connected electrically to a ground 26, but to which no electrical contact need be made in this case. Preferably, the lower stage 16, which may be formed of e.g. aluminum, provides a surface 28 for supporting a component of a medicinal inhalation device, a medicinal inhalation device or a part of such a device including a surface onto which a thin film is to be deposited, referred to in the following as the substrate 54. The lower stage 16 is preferably maintained at a temperature lower than that of the hot-filament such that reactive species produced in the vicinity of the filament are transported to the substrate 54, where they deposit and polymerize. Cooling coils 31 connected to a coolant loop 30 and a temperature controller 32, or other appropriate cooling mechanism, can be employed to maintain a substrate 54 supported on the lower stage 16 at a desired temperature. Depending on the chamber configuration the upper shower-head and the lower stage are preferably spaced apart by, about 0.5 cm to about 30.5 cm.

The substrate can be singly or batch processed. In a single-substrate process, the substrate is supported on the lower stage 16. In a multi-substrate process, a plurality of substrates can be suspended in the vacuum chamber between the upper shower head and lower stage by way of, e.g., an aluminum substrate boat, vertically supported by, e.g., a chamber sidewall anchor, and having support slots for holding substrates in a desired configuration. Preferably, the selected multi-substrate support configuration enables a user to adjust individual substrates' positions without substantial complexity; such substrate position adjustment may be desirable at intervals during a deposition process for enhancing deposition uniformity across the span of a substrate. As will be recognized by those skilled in the art, a support structure, e.g., a bulk PTFE block, or other support structure, can be employed to accommodate a specific substrate in the deposition chamber. The only requirement imposed by the thermal-CVD process on structural supports is the ability to maintain a supported substrate at a desired temperature that is lower than the pyrolysis temperature. Examples of suitable support structures are disclosed in WO 97/42356. In addition, reorientation of a substrate and its support structure can be enabled by, e.g., manual reorientation during the deposition process, or e.g. a spinning or rotating holder, can be designed-in as a mechanism integral to the support structure. Substrate reorientation techniques, as are routinely employed in industrial vapor deposition and ion-implantation processes, can correspondingly be employed to enhance deposition uniformity.

An initial adhesion-promotion sub-step can be employed prior to the deposition process to enhance and promote adhesion of the depositing species on a given substrate. For example, an adhesion promoter can be spin-applied to a planar substrate or sprayed on to a complex geometrical substrate. Alternatively, an adhesion promoter can be vapor-deposited in situ in the deposition chamber just prior to the fluorocarbon polymer film deposition process. Examples of suitable adhesion promoters include 1H, 1H, 2H, 2H-Perfluorodecyltriethoxysilane; 1H, 1H, 2H, 2H-Perfluorooctyltriethoxysilane; 1H, 1H, 2H, 2H-Perfluoroalkyltriethoxysilane; perfluorooctyltriclorosilane; all classes of vinyl silanes, as well as other adhesion promoters, as will be recognized by those skilled in the art.

Thermal excitation mechanisms other than a hot-filament are equally suitable for the thermal-CVD process. Indeed, it is preferable that the selected thermal mechanism, together with the gas delivery system, provide both uniform gas input and uniform pyrolysis of the gas. Hot windows, electrodes, or other surfaces, as well as heated walls of the deposition chamber, can alternatively be employed in pyrolysis configurations aimed at producing uniform gas pyrolysis.

In one alternative, the upper shower-head 14 is itself heated, whereby input feed gas is pyrolyzed as it passes through the shower-head. Such heating can be accomplished by, e.g., applying a dc voltage to the shower-head, which preferably consists of, e.g., aluminum or stainless steel. As the input feed gas is delivered from the feed gas source to the heated upper shower-head, the gas preferably is maintained at a temperature at which it is not pyrolyzed, such that substantially all pyrolysis occurs only once the gas enters the shower-head. In this case, of course, an additional hot-filament is not needed in the deposition chamber.

In a similar alternative, the upper shower-head 14 is outfitted with an array of tubes, each shower-head hole having a tube protruding from it. Such tubes consist of, e.g., anodized aluminum or stainless steel In this case, the shower-head is not itself heated. Instead, the tubes protruding from the shower-head are configured to fit into a corresponding array of holes in a heated plate suspended just below the shower-head, such that the tubes extend to some depth, e.g., substantially through, the holes in the plate. In this configuration, gas in the shower-head passes from the shower-head through the tubes and through the heated plate, whereby the gas is heated at the lower surface of the plate as it exits the tubes. This produces a plane of pyrolyzing gas that is substantially parallel to the lower electrode. As a result, both uniform gas injection and uniform pyrolysis of gas is achieved. Accordingly, this enables production of a substantially uniform reactive gas species environment in the vicinity of substrate to be coated by the deposition process. As will be recognized by those skilled in the art, this pyrolysis configuration is therefore preferable for applications in which deposition uniformity is of importance, e.g., in the case of deposition on a large substrate, or a spaced array of substrates in, e.g., a production environment. Preferably, the tubes connected between the shower-head and the heated plate are slightly smaller in diameter than the holes in the heated plate, such that no substantial pyrolysis occurs until the gas exits the tubes and at the plate lower surface. Additionally, the heated plate is preferable suspended slightly below the shower-head such that the shower-head is not substantially heated by the plate. The plate is preferably formed of, e.g., aluminum or stainless steel, is thick enough to produce uniform heating, and is heated by conventional techniques.

In another alternative technique, the input feed gas is heated in, e.g., a gas delivery tube 56 connecting the gas feed source 18 to the upper shower-head 14. Here, the pyrolyzed gas is preferably piped to the location of the substrate to be coated in a manner similar to that for conventional downstream-ashing processes. In yet another pyrolysis configuration, a cold feed gas is mixed with a hot inert gas, such as argon, in the deposition chamber. In this case, the inert gas is injected and heated by, e.g., one of the processes described above. Mixture of the heated gas with the cold input feed gas results in pyrolysis of the feed gas. This pyrolysis technique has the advantage of eliminating a pyrolysis surface in the chamber that itself is coated with the reactive gas species produced by the pyrolysis. Other direct heating techniques, e.g., laser heating techniques, can also be employed, as can be employed, in general, a wide range of other pyrolysis mechanisms.

Any suitable feed gas can be used that provides a monomer which can be pyrolyzed to provide difluorocarbene species (CF₂) for producing a fluorocarbon polymer film having a high fraction of CF₂ groups and a low degree of polymer cross-linking. Example monomers for use as a deposition feed gas include C₂F₄, C₃F₈, CF₃H, CF₂H₂, CF₂N₂ (difluordiaxirine), CF₃COCF₃, CF₂ClCOCF₂Cl, CF₂ClCOCFCl₂, CF₃COOH, difluorohalomethanes such as CF₂Br₂, CF₂HBr, CF₂HCl, CF₂Cl₂, and CF₂FCl; difluorocyclopropanes such as C₃F₆, C₃F₄H₂, C₃F₂Cl₄, C₂F₃Cl₃, and C₃F₄Cl₂; trifluoromethylfluorophosphanes such as (CF₃)₃PF₃, (CF₃)₂PF₃, and (CF₃)PF₄; or trifluoromethylphosphino compounds such as (CF₃)₃P, (CF₃)₂P—P(CF₃)₂, (CF₃)₂PX, and CF₃PX₂, where X is F, Cl, or H. Other monomers can also be employed.

One preferable monomer is hexafluoropropylene oxide (C₃F₆O or HFPO). HFPO is characterized by a highly-strained epoxide ring and is understood to decompose under pyrolysis to form a fluorinated ketone and the desired difluorocarbene. The fluorinated ketone is relatively stable, compared with the difluorocarbene. This is understood to lead to a high CF₂ content in a film as polymerization occurs at the film deposition surface. Oxygen present in the monomer is tied up in the relatively unreactive ketone decomposition by-product, whereby little oxygen is incorporated into the film.

The flow rate of the monomer feed gas typically ranges between about 1 and 200 standard cubic centimeters per minute (seem), and 30 seem is preferable for the monomer HFPO. Inert gases such as nitrogen or argon can be added to the monomer feed gas; preferably, no inert gas is included with the monomer HFPO, however.

It is also preferable that the partial pressure of the reactive species be kept to a low level that prevents homogeneous gas-phase reactions, which could cause particle production in the gaseous environment rather than on the substrate surface to be coated. The pressure of the vacuum deposition chamber can be set at a pressure of between about 1 milliTorr to 50 Torr during the deposition process, with a pressure of about 0.3 to 1.2 Torr being preferable.

The substrate surface onto which a film is being deposited is held during the deposition substantially at a temperature lower than that of the heat source to induce deposition and polymerization of the CF₂ species on the surface, preferably at a temperature less than about 300 K. Preferably the heat source temperature is greater than about 500K, more preferably greater than 600 K.

Through the selected of appropriate process conditions, the film deposition rate can be optimized so that desired film thickness can be provided in reasonable and practical processing times. For example, a deposition rate of about 1.8 μm/hour can be achieved by using the following process conditions for the deposition of a fluorocarbon polymer film onto a substrate surface: Flowing 12.5 seem of undiluted HFPO (from PCR, Inc.) of about 99% purity, into a parallel plate vacuum deposition chamber, like that described above, with a hot-filament of Ni/Cr wire positioned under the upper shower-head and substrate supported on the lower stage, while maintaining the chamber at a pressure of about 1 Torr, the hot-filament at a temperature of about 673K using a de voltage and the lower stage at a temperature of about 293±3K.

It is preferred that the resulting fluorocarbon polymer film has a thickness of about 0.0002 μm to about 100 μm, more preferably about 0.01 μm to about 3 μm.

Thermal post-deposition steps can be carried out in situ in the deposition chamber. For example, a post-deposition annealing in air, or in nitrogen or other inert gas, can be employed for, e.g. film stress relief, dangling bond passivation, or thermal stability enhancement. Such annealing can be carried out at a temperature of between about 50° C. and 400° C.

In order to enhance potentially desirable customization of the properties of a film in situ during film deposition, a thermal CVD process may be combined with a plasma enhanced CVD (“PECVD”) process, either a continuous- or pulsed-PECVD.

A vacuum deposition chamber like that schematically illustrated in FIG. 1 can be employed for carrying out such hybrid deposition processes. In particular for PECVD processes, as mentioned above and as shown in FIG. 1, the upper shower-head 14 may be a powered electrode, which is connected electrically to a radio frequency (rf) power source 24, or other suitable power source, for producing a plasma of the feed gas in the chamber, while the lower stage 16 may be a ground electrode, which is connected electrically to a ground 26 of the vacuum chamber system.

A plasma is provided by applying continuously or preferably pulsing a suitable plasma excitation (preferably rf excitation) to the feed monomer gas introduced into the deposition chamber. In particular for pulsed-PECVD, the rf or other plasma excitation power is alternately turned on and off with a desired duty cycle. For such pulsed-PECVD processes, the plasma excitation on-time is preferably between about 100 microsecond and 1 second (more preferably between 1 millisecond and 100 milliseconds), and the plasma excitation off-time is between about 100 microsecond and 1 second (more preferably between 150 milliseconds and 1150 milliseconds). Even more preferably, the plasma excitation on-time is about 5 to about 15 milliseconds (most preferably about 10 milliseconds) and the plasma excitation off-time is about 200 to about 1000 milliseconds (most preferably an off-time of about 200 to about 400 milliseconds). The plasma excitation power is preferably in the range of about 50 to about 350 Watts for a 11.5 cm diameter grounded electrode surface, with the power more preferably being about 270 to 310 Watts. For rf plasma excitation, the rf plasma excitation frequency is set at, e.g., about 13.56 MHz, as is conventional for plasma processes, but other frequencies can be employed.

During PECVD processes, the substrate onto which a (CF₂)ₙ-like film is to be deposited is preferably held at an electrical potential to induce deposition and polymerization of the CF₂ species on the surface, preferably at an electrical potential of between about −400 V to +400 V, including a ground potential or floating potential at the potential of the plasma. Ground potential or floating potential is preferred. Also the substrate onto which a film is being deposited is typically held at a temperature during the deposition to induce deposition and polymerization of the CF₂ species on the surface, preferably at a temperature of between about −40° C. and +200° C.; more preferably at about 293 K.

In regard to other process conditions and suitable monomers for PECVD processes, those described above for thermal CVD process can be applied.

In a first such hybrid process, two or more deposition intervals are defined, each interval employing one or both PECVD and thermal-CVD conditions. For example, during a deposition initiation interval, one of continuous- or pulsed-PECVD conditions are provided; then during a growth phase interval thermal-CVD conditions alone or in combination with PECVD conditions are provided. In intervals during which continuous- or pulsed-PECVD conditions alone are provided, it is preferred that the monomer gas is not substantially pyrolyzed during plasma excitation power application.

Processes such as this example two-interval process provide several advantages. First, it is recognized that ion bombardment, which takes place during continuous- and pulsed-PECVD, aids in initiation of deposition of gas species onto a substrate surface, due, e.g., to the electric field bias inherent in the plasma excitation conditions. This in turn enhances the adhesion of the depositing film to the underlying substrate surface. Thus, the ion, neutral, and free radical production provided by a plasma process can be advantageously employed at the start of the deposition to aid in film nucleation and to enhance film adhesion. The film surface roughness characteristic of PECVD deposition conditions also aids in adhesion. Although the pulsed-PECVD process conditions produce superior results, continuous-PECVD conditions can also be employed for this initiation interval.

During a sequential growth phase, the plasma is extinguished and the gas heating is commenced, whereby only thermal processes produce the reactive gas species that polymerize on the surface of the substrate. During the thermal-CVD growth interval, the properties of the film can be further customized. For example, a plasma can be ignited for one or more brief sub-intervals to reduce film crystallinity, or to otherwise modify the film characteristics in a depth-dependent manner. Alternatively, a relatively low-power plasma can be maintained during either the entire duration or a portion of the growth interval duration. As in the initiation interval, either continuous- or pulsed-PECVD can be employed in either alternative.

For such hybrid processes in which thermal-CVD conditions are provided in combination with PECVD conditions during a growth phase interval, the CF₂ content of the deposited film surface can be desirably maximized by following the growth phase interval with a final deposition interval during which thermal CVD conditions alone are provided until the end of the deposition cycle.

The two or three-interval deposition processes just described can be employed to produce a graded interface between an underlying substrate and a fluorocarbon film having characteristics which vary with depth through the film. As will be recognized by those skilled in the art, other combinations of thermal-CVD and PECVD conditions can be employed to achieve customization of a (CF₂)ₙ-type polymer thin film as the film is deposited.

Also as will be recognized by those skilled in the art, methods of making a medicinal inhalation device in accordance with the invention may include other steps, e.g. assembling the device and/or containing a medicinal formulation within the device. It will also be appreciated that the step of forming a (CF₂)ₙ-type polymer thin film is typically performed prior to the final assembly of the device and containment of a medicinal formulation within the device. Also in regard to method of making a component for a medicinal inhalation device in accordance with the invention, those skilled in the art will appreciate the method may comprise other steps, e.g. injection molding, deep drawing, cleaning operations and other surface preparation processes.

Methods in accordance with the invention advantageously allow the provision of medicinal inhalation devices or components therefor comprising a fluorocarbon polymer thin film on a surface of the device or the component; wherein the film exhibits advantageously high CF₂ fraction (preferably at least about 85%) in conjunction with a low degree of cross-linking (preferably less than about 20%). The deposited films are characterized as smooth, conformal, coatings that exhibit sufficient flexibility to withstand mechanical bending of a three-dimensional structure, e.g. a valve spring, or mechanical compression of a structure, e.g. elastomeric seals, on which they are deposited. The films exhibiting properties that closely resemble those of bulk PTFE show desirably high chemical stability and inertness resisting degradation upon exposure to various medicinal formulations, in particular liquefied propellant-based medicinal inhalation formulations.

In more preferred embodiments, the deposited films exhibit a CF₂ fraction of at least about 90% and most preferably of at least about 95%. The deposited films are also more preferably characterized by cross-linking density of less than 15% and most preferably less about 10%. Due to the high CF₂ fraction and the low degree of cross-linking, the fluorine to carbon ratio (F/C ratio) of deposited films approaches that of bulk PTFE, preferably being from about 1.4:1 to about 2.2:1, more preferably 1.6:1 to 2.2:1, preferably as determined by X-ray photoelectron spectroscopy (XPS).

CF₂ fraction and the degree of cross-linking of the deposited films are preferably determined by high resolution carbon-1s XPS. In a carbon-1s XPS spectrum of a fluorocarbon film (typically measured over a binding energy range of 298 to 280 eV), carbon-1s binding energies are assigned as follows: 294 eV (CF₃), 292 eV (CF₂), 289.5 eV (CF), 287.5 eV (C—CFₙ). In particular, the spectrum peak at 292 eV is indicative of a CF₂ bonding environment, and deconvolution of the spectrum allows a determination of the area of the CF₂ peak relative to total area of the spectrum and thus the percentage of CF₂ fraction.

The percentage of cross-linking in the two films is also determined based on deconvolution of a carbon-1s XPS spectrum of a film. In this determination, the number of network-forming bonds of the XPS-resolvable CF₃, CF₂, CF, and C—CFₙ groups are taken to be 1, 2, 3, and 4, respectively. This assignment assumes that the number of carbon-carbon double bonds is small. Of these four resolvable groups, only the groups having more than 2 bonds are considered as cross-linkable, i.e., able to form a network; thus, the CF and C—CFₙ groups are characterized as crosslinkable. Accordingly, the compositional cross-linking percentage of a given film can be determined by the XPS deconvolution area of these two groups relative to the total area of the spectrum.

The deposited film also typically exhibit a desirably low density of residual unpaired electrons, preferably about 10¹⁸ spins/cm³ or less, as preferably determined by Electron Spin Resonance Spectrometry (ESR).

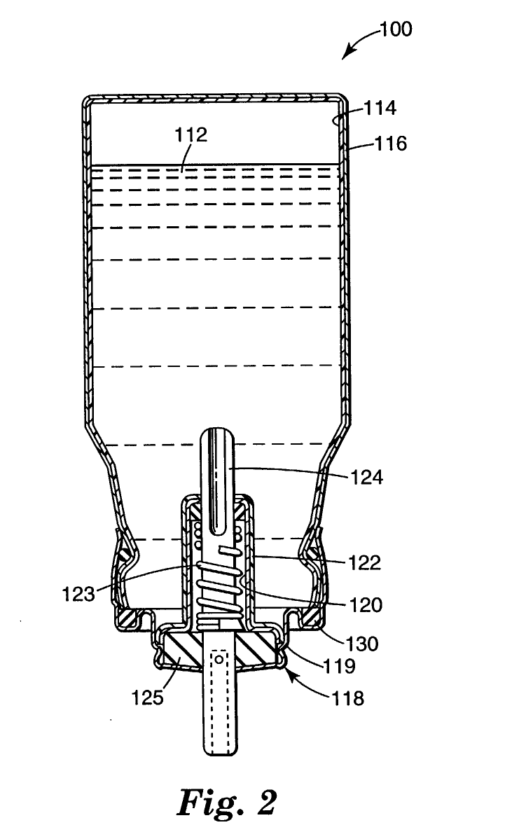

In preferred methods of making a medicinal inhalation devices, a fluorocarbon (CF₂)ₙ-type polymer thin film is formed on an internal surface of the device, more preferably on an internal surface that is to be in contact with a medicament or a medicinal formulation during storage or delivery from the device. Also, due to the desirable mechanical stability of the deposited films, it is advantageous to form the film on a surface that comes in contact with a movable component of the device or on a surface of a movable component of the device. Similarly, in preferred methods of making a component for a medicinal inhalation device, the component is a movable component of such a device and/or a component having a surface which will come into contact with a movable component of such a device and/or a component having a surface which will come in contact with a medicament or a medicinal formulation during storage in or delivery from a medicinal inhalation device. This can be better understood by reference to the accompanying drawings, where FIGS. 2, 3, 4 and 5 show preferred embodiments of medicinal inhalation devices and/or components therefor provided in accordance with the invention.

FIG. 2 shows a cross-sectional view of a preferred medicinal inhalation device 100 comprising a pressurizable aerosol container 116 equipped with a metering valve 118. Such a device is typically used for the delivery of propellant-based medicinal inhalation formulations. A fluorocarbon (CF₂)ₙ-type polymer thin film 114, 122 may be advantageously formed on an internal surface of the container 116 and/or on a surface of the metering chamber 120.

Although not shown in FIG. 2, it is preferred that as many other surfaces in contact or surfaces that will come in contact with the formulation 112 as feasible are deposited with a fluorocarbon polymer thin film. Such surfaces can include for example one or more surfaces of the metering chamber 120, the ferrule 119, the gasket seal 130, the valve stem 124, valve seal 121, valve spring 123 and/or diaphragm 125. The formation of a fluorocarbon polymer thin film on one or more surfaces of a movable component, e.g. valve stem 124, or on one or more surfaces of a component, e.g. a valve seal 121, valve spring 123 and/or diaphragm 125, coming into contact with such a movable component, allows the additional advantage of reduction of surface energy between the correspondingly components and thus enhanced operation of the valve and device. In regard to dispensing valves, such as a metering valve 118 shown in FIG. 2, it is advantageous to form a (CF₂)ₙ-type polymer thin film on all the product contact surfaces of the individual components making up the valve and all surfaces which form sliding seals such as the valve stem 124 and the diaphragm 125 and valve seal 121.

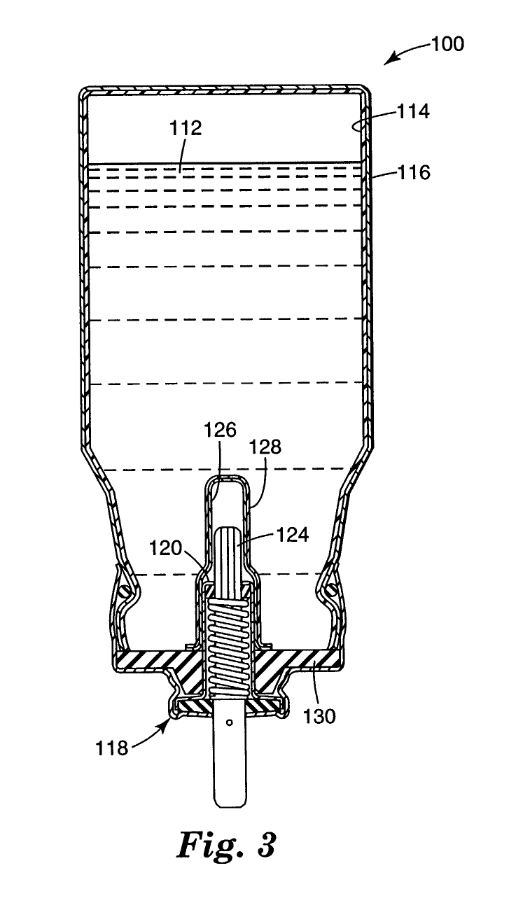

FIG. 3 shows an alternative preferred medicinal inhalation device that is essentially the same as that illustrated in FIG. 2, but comprises a retaining cup 126, onto which a fluorocarbon polymer thin film is deposited 128.

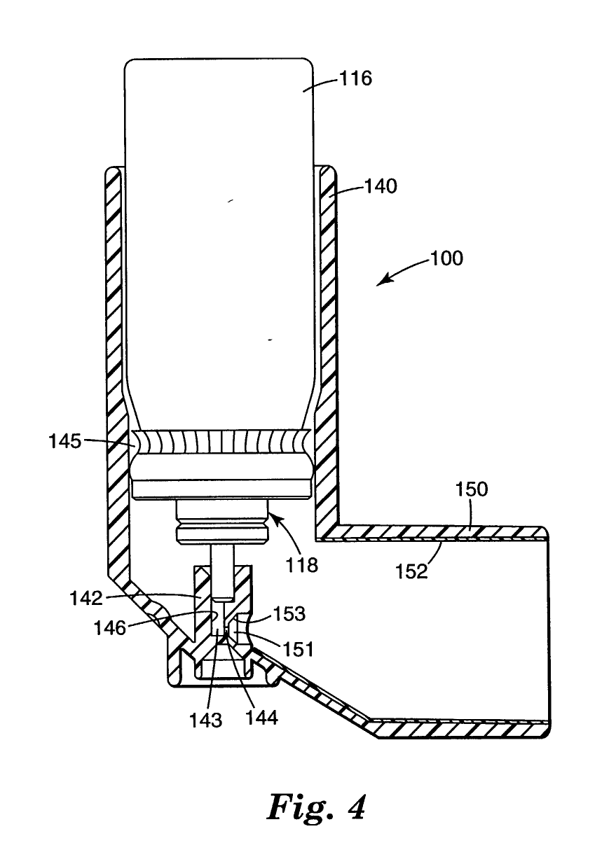

FIG. 4 shows a cross-sectional view of an another preferred medicinal inhalation device 100 comprising a pressurizable aerosol container 116 equipped with a valve 118. The device 100 also includes a housing 140 for receiving the container 116 and an actuator 150 for insertion into the mouth of a user of the device 100. The lower wall 141 of the housing includes an annular stem socket 142 for receiving the valve stem of the valve 118. The socket communicates via a duct 143 ending in an atomization orifice 144 to a cone region 151 of the actuator 150. A passage 145 between the aerosol container and the housing allows air to flow through the housing to the actuator outlet. To minimize deposition during delivery, a fluorocarbon (CF₂)ₙ-type polymer thin film 152 may be advantageously formed on an internal surface of the actuator 150. Although not shown in FIG. 4, it is also particularly desirable to form a (CF₂)ₙ-type polymer thin film on a surface 153 of the cone region 151 of the actuator, an internal surface 146 of the actuator stem socket 142, and/or on a surface of the orifice 144.

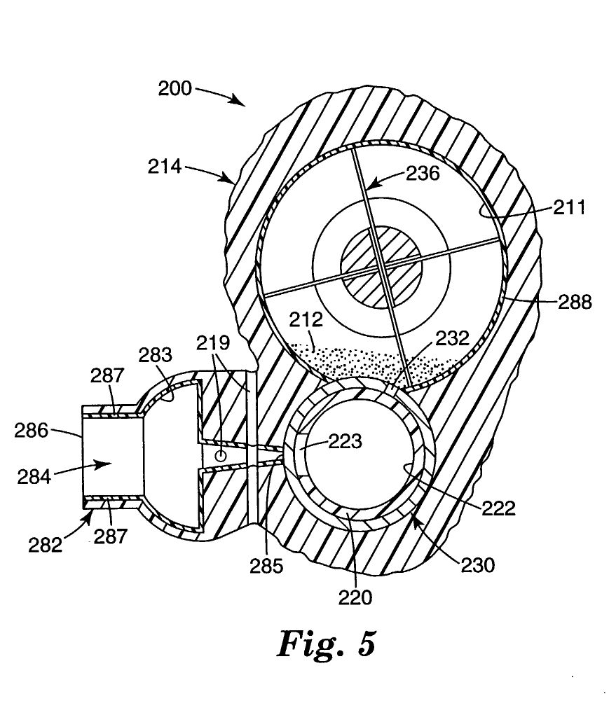

FIG. 5 shows a cross-sectional view of yet another preferred medicinal inhalation device. The device 200 illustrated in FIG. 5, a DPI-type of medicinal inhalation device, facilitates oral inhalation of powdered medicaments 212 that are stored in bulk form in medicament reservoir 211 defined by a housing 214. The device comprises a powder loading blade 236 for providing an agglomerated, predetermined dose of powdered medicament within a dosage chamber 232 of a movable dosage member 230 and a pressurization assembly including an inner housing 220 forming a gas pressure chamber 222, a pressure outlet passageway 223 and a pressurization member (not shown) for providing a deagglomeration pressure. For passage of a respirable dose of dry powder for subsequent delivery to the user, a medicament delivery passageway 284, including an optional declaration portion 283, extends between an injection inlet opening 285, which intermittently communicates with the dosage chamber 232, and an outlet opening 286 through a mouthpiece portion 282. One or more air entrance passageways 219 facilitate the passage of a respirable dose of dry powder along the medicament delivery passageway. A fluorocarbon (CF₂)ₙ-type polymer thin film 287, 288 may be advantageously formed on a surface of the medicament delivery passageway 284 and/or a surface of the medicament reservoir 211. Although not shown in FIG. 5, it is preferred one or more surfaces of the dosage chamber 232, the movable dosage member 230, the powder loading blade 236, and/or the inner housing 220.

As an example, a coating according to the present invention was applied to stainless steel type 305 deep drawn metering chamber components of a metering valve (of the type corresponding to element 122 of FIG. 2), manufactured by 3M Neotechnic Ltd. The components were first degreased by washing with 1,1,1-trichloroethane prior to use, and then coated with a layer of PTFE using the apparatus of FIG. 1.

Although there are different ways to assess the coating effectiveness, in this case the effectiveness was assessed using Brilliant Blue FCF (as a pigmented model of drug) supplied by Warner Jenkinson Europe, which was micronised by passing through a 2 inch fluid energy mill. The micronised Brilliant Blue dye (300 milligrams) was placed in a 50 ml glass beaker with 3 coated metering chamber components and 3 uncoated metering chamber components. The beaker was tilted to an angle of about 30 degrees from vertical and was rotated gently for one minute.

Each component was individually removed from the beaker by using tweezers, holding the component by the flange, and immersed in n-heptane (50 ml) contained in a second beaker. This was agitated linearly for 5 seconds using 10 movement cycles. The coated and uncoated components were then placed in 2 separate Petri dishes and allowed to dry.

Each component was then washed with distilled water in a beaker to dissolve the dye, and the washings quantitatively transferred into a 25 ml volumetric flask. A Perkin Elmer Lambda 20 uv/visible spectrophotometer was used to obtain a calibration with standard solutions of Brilliant Blue dye, using the following conditions:

| | |
|---|---|
| Measurement wavelength | 629.0 nm |
| Baseline correction wavelengths | 480.0 nm and 720.0 nm |
| Response | 2 seconds |
| Slit width | 1.0 nm |
| Smooth points | 0 |
| Curve type | Linear |

Samples from the volumetric flasks were diluted by a factor of 50 prior to analysis in the spectrophotometer. The Brilliant blue dye washed from each component was quantified in terms of micrograms per component.

Results

| Sample | Brilliant Blue deposited (mcgs per component) |
|---|---|
| Uncoated | 1987 |
| | 2112 |
| | 1486 |
| Coated | 1019 |
| | 1363 |
| | 1276 |

These results show, on average, a 35% reduction in deposition of dye on the coated components.

Methods in accordance with the invention can also be used to provide other medicinal inhalation devices including nebulizers, pump spray devices, nasal pumps, non-pressurized actuators, breath actuated inhalers and breath-coordinating devices, spacers, dose counters, or components for such devices.

1. A method of making a medicinal inhalation device comprising a step of forming a fluorocarbon (CF₂)ₙ-type polymer thin film on a surface of the device, said forming step comprising the sub-steps of:
a) exposing a monomer gas to a source of heat having a temperature sufficient to pyrolyze the monomer gas, the monomer gas selected to produce upon pyrolysis a source of reactive species that includes polymerizable CF₂ species and that selectively promotes CF₂ polymerization, the reactive species source being in the vicinity of the surface on which the fluorocarbon polymer film is to be formed; and
b) maintaining the surface substantially at a temperature lower than that of the heat source to induce deposition and polymerization of the CF₂ species on the surface.

2. A method of making a component for a medicinal inhalation device comprising a step of forming a fluorocarbon (CF₂)ₙ-type polymer thin film on a surface of the component, said forming step comprising the sub-steps of:
a) exposing a monomer gas to a source of heat having a temperature sufficient to pyrolyze the monomer gas, the monomer gas selected to produce upon pyrolysis a source of reactive species that includes polymerizable CF₂ species and that selectively promotes CF₂ polymerization, the reactive species source being in the vicinity of the surface on which the fluorocarbon polymer film is to be formed; and
b) maintaining the surface substantially at a temperature lower than that of the heat source to induce deposition and polymerization of the CF₂ species on the surface.

3. A method of claim 1 or claim 2, wherein the monomer gas comprises hexafluoropropylene oxide.

4. A method of claim 1 or claim 2, wherein the heat source to which the monomer gas is exposed comprises a resistively-heated conducting filament suspended above the surface.

5. A method of claim 1 or claim 2, wherein the heat source to which the monomer gas is exposed comprises a heated plate having a pyrolysis surface that faces the surface on which the fluorocarbon polymer film is to be formed.

6. A method of claim 1 or claim 2, wherein the heat source temperature is greater than about 500 K and wherein sub-step b) comprises maintaining the surface is at a temperature less than about 300 K.

7. A method of claim 1 or claim 2, wherein the forming step further comprises a first sub-step of applying plasma excitation power to the monomer gas.

8. A method of claim 7, wherein the monomer gas is not substantially pyrolyzed during plasma excitation power application.

9. A method of claim 7, wherein the applied plasma excitation power is characterized by an excitation duty cycle having alternating intervals in which excitation power is applied and in which no excitation power is applied to the monomer gas.

10. A method of claim 1 or claim 2, wherein the sub-step a) further comprises simultaneous application of plasma excitation power to the monomer gas.

11. A method of claim 10, wherein the applied plasma excitation power is characterized by an excitation duty cycle having alternating intervals in which excitation power is applied and in which no excitation power is applied to the monomer gas.

12. A medicinal inhalation device comprising a fluorocarbon (CF₂)ₙ-type polymer thin film on a surface of the device, said film having a CF₂ fraction of at least 85% and a cross-linking density of less than about 20%.

13. A medicinal inhalation device of claim 12, wherein said film has a CF₂ fraction of at least 90% and a cross-linking density of less than about 15%.

14. A medicinal inhalation device of claim 13, wherein said film has a CF₂ fraction of at least 95% and a cross-linking density of less than about 10%.

15. A medicinal inhalation device of any one of claims 12 to 14, wherein said film has a thickness of about 0.0002 μm to about 100 μm.

16. A component for a medicinal inhalation device comprising a fluorocarbon (CF₂)ₙ-type polymer thin film on a surface of said component; said film having a CF₂ fraction of at least 85% and a cross-linking density of less than about 20%.

17. A component for a medicinal inhalation device of claim 16, wherein said film has a CF₂ fraction of at least 90% and a cross-linking density of less than about 15%.

18. A component for a medicinal inhalation device of claim 17, wherein said film has a CF₂ fraction of at least 95% and a cross-linking density of less than about 10%.

19. A component for a medicinal inhalation device of any one of claims 16 to 18, wherein said film has a thickness of about 0.0002 μm to about 100 μm.
